(19) United States
(12) Patent Application Publication
(10) Pub. No.: US 2004/0115802 A1
(43) Pub. Date: Jun. 17, 2004

(54) Cargo loading and unloading systems

(76) Inventors: Gregory John Dickson, Tauranga (NZ); Bruce Walton, Auckland (NZ)

Correspondence Address: YOUNG & THOMPSON, 745 SOUTH 23RD STREET 2ND FLOOR, ARLINGTON, VA, 22202

(21) Appl. No.: 10/468,756

(22) PCT Filed: Feb. 22, 2002
(86) PCT No.: PCT/NZ02/00019

(30) Foreign Application Priority Data
Feb. 23, 2001 (NZ) 510133

(51) Int. Cl. C12N 5/06

(52) U.S. Cl. 435/326

(57) ABSTRACT

A graphical user interface is provided for providing a three dimensional representation of a cargo storage space and a three dimensional representation of a cargo item to be placed within that storage space. The interface allows the representation of the cargo item to be moved to different positions in the storage space within the constraints of the real dimensions of the storage space and of the cargo item. Therefore a user may use the interface to plan the storage of cargo within a certain space such as the hull of a ship or aircraft. In this way, efficiency of cargo storage can be improved. Methods for determining the shape of a number of cargo items in a selected space, and for determining whether or not a cargo item of a given shape will fit within a predetermined space are also provided.

### FIELD OF THE INVENTION

This invention relates to means for the planning, management and recording of loading or unloading processes for cargo items to or from cargo carrying vessels including ships, containers, aircraft, trains or road vehicles.

### BACKGROUND OF THE INVENTION

Cargo loading and unloading tasks often require complex planning and can be difficult to perform. The tasks are made difficult by different items of cargo having different shapes and different behavioural characteristics when loaded into storage spaces. In particular, the hulls of vessels such as ships and aircraft often have highly variable shapes which makes planning the loading of the ship even more difficult.

### OBJECT OF THE INVENTION

It is an object of the present invention to provide improved cargo loading or unloading systems or methods and apparatus, or to at last provide the public with a useful choice.

### SUMMARY OF THE INVENTION

In one aspect the invention consists in a graphical user interface for indicating the location of one or more cargo items in at least one storage space, the interface including;

a three dimensional representation of the storage space and three dimensional representation of the one or more cargo items in that storage space, and whereby the representation of the one or more cargo items is able to be moved to a desired location within the representation of the storage space and whereby the representation of the one or more cargo items is representative of the real space required for location of the one or more cargo items in the real storage space.

Preferably the representation of the one or more cargo items is shown to conform to the representation of the storage space dependent upon the physical properties of the one or more cargo items.

The storage space may be a physically enclosed space but does not need to be a physically enclosed space, so it may include an area on a wharf for example, or on the deck of a ship, or open floor space in a warehouse or shed.

Data relating to the representation may provide the weight, volume or number of items for the one or more cargo items. The spatial position of the one or more items within the cargo space is also known. Therefore the invention encompasses analysis of the stability of the vessel.

In a further aspect the invention consists in a method for determining the shape of a predetermined number of elongate cargo items of substantially uniform dimensions in a storage space, the method including steps of

determining the shape at a cross section through the storage space at or adjacent to one end of the elongate cargo items when placed in the storage space,

determining the shape at a cross section through the storage space at or adjacent to the other end of the elongate cargo items when placed in the storage space,

superimposing the shapes to determine a polygon the sides of which bound the common region of the superimposed shapes,

whereby the shape of the cargo items corresponding to the polygon in two dimensions and the length of the elongate items in the third dimension.

Preferably the cargo items comprise logs.

In a further aspect the invention consists in a method for determining the shape of at least one cargo item having at least one fixed dimension in a storage space, the method including the steps of

determining the shape at a cross section through the storage space at or adjacent to the beginning of the fixed dimension of the cargo item when placed in the storage space,

determining the shape at a cross section through the storage space at or adjacent to the other end of the fixed dimension of the cargo item when placed in the storage space,

superimposing the shapes to determined a polygon the sides of which bond the common region of the superimposed shapes,

whereby the shape of the cargo items corresponding to the polygon in two dimensions and the fixed dimension in the third dimension.

The invention includes graphical representations of the shape of the cargo item. Data relating to the representation may provide the weight, volume or number of items for the one or more cargo items. The spatial position of the one or more items within the cargo space is also known. Therefore the stability of the vessel may be analysed.

In a further aspect the invention consists in a method for determining whether a cargo item may be placed within a storage space, the method comprising the steps of

for at least one dimension of the item,

determining whether a first end of the item in the relevant dimension is within the storage space,

if the first end of the item is within the cargo storage space, then determining whether the second end of the item is within Me cargo storage space.

In a further aspect the invention consists in a method for providing a indication of the predicted stability of a vessel when loaded with one or more cargo items, the method including the steps of determining the spatial position of each item, providing information relating to the mass of each item, determining a spatial position for a centre of mass for the cargo, and using the spatial position of the centre of mass to provide an indication of the stability of the loaded vessel.

Preferably the information relating to mass is derived from one or more of the number of items, the volume of the items, density, or mass.

Preferably the vessel comprises a ship, or a hold in a ship.

Preferably the spatial position and magnitude of the centre of mass is compared with one or more characteristics of the vessel to provide an indication of stability of the vessel,

In a further aspect the invention consists in a method for determining whether a cargo item may be placed within a storage space, the method comprising the steps of

for at least one dimension of the item,

determining the shape of the item and the shape of the storage space at a cross section through the storage space at or adjacent to the relevant dimension of the cargo item when placed in the storage space,

determining the shape of the item and the shape of the storage space at a cross section through the storage space at or adjacent to the end of the relevant dimension of the cargo item when placed in the storage space, and

if any part of the item shape exceeds the storage space shape, then providing an indication that the item will not fit within the space.

Preferably the storage space includes an available space within a larger storage area

In a further aspect the invention consists in an interface for planning loading or unloading of one or more cargo items in the storage space of a cargo carrying vessel, the interface including a first two dimensional representation of the one or more cargo items in the cargo space, viewed from the first direction, and a second two dimensional representation of the one or more cargo items in the storage space viewed from another direction substantially perpendicular to the first direction, movement of the representation of the one or more cargo items in the first representation resulting in corresponding movement in the second representation.

Preferably the first two dimensional representation is provided in a first window or fame, and the second two dimensional representation is provided in a second window or frame.

In a further aspect the invention consists in a method of planning or monitoring loading or unloading of cargo or from a cargo carrying vessel, including the steps of

providing a representation of a storage space

providing data relating to at least one cargo item

selecting a position for the cargo item in the storage space

comparing the dimensions of the cargo item and the selected position of the item in tho space, and

if the cargo item will not fit in the selected position either providing an indication to the user and/or repositioning the cargo item to fit within the storage space.

In a further aspect the invention consists in a method of planning or monitoring loading or unloading of cargo to or from a cargo carrying vessel, including the steps of

providing data relating to a storage space

providing data relating to a cargo item

providing data relating to a selected position of the cargo item in the space,

determining a measurement representative of the stability of the vessel with the cargo item in the selected position, and comparing the measurement with a predetermined value indicative of allowable stability or instability.

In a further aspect the invention consist in a computer or network of computers programmed or otherwise operable to effect the method(s) and/or interfaces of any one or more of the preceding statements.

To those skilled in the art to which the invention relates, many changes in constructions and widely different embodiments and applications of the invention will suggest themselves without departing from the scope of the invention as defined in the appended claims. The disclosure and descriptions herein are purely illustrative and are not intended to be in any sense limiting.

The invention consists of the foregoing and also envisages constructions of which the following gives examples only.

### DRAWING DESCRIPTION

FIG. 1 is a flow diagram of a process flow according to the invention

FIG. 2 is a flow diagram of A cargo item shape determining process according to the present invention.

FIGS. 3 to 13 are screen shots of an interface according to the present invention illustrating some of the capabilities of the interface in use.

### DESCRIPTION OF PREFERRED EMBODIMENT

This invention relates to a further method or interface and/or further improvements to the method and interface described in our co-pending application number PCT/NZ00/00058 (WO0063805), the contents of which are specifically incorporated herein by reference in their entirety.

As described in our co-pending application, the method and interface of the present invention may be implemented on a computer or network of computers or other processing machines having appropriate display means. For convenience, known computers on which the invention may be implemented are not described in detail herein. To those skilled in the art, such computers and suitable operating systems are well known.

In the examples below, reference is made to cargoes of logs being loaded or unloaded to or from a ship. This is for the purposes of illustration. The cargo may comprise many different materials or products, and the applicable transport vehicles are not limited to ships. Nor does the planned cargo location need to be the hold of a ship, or any physically enclosed space. Designated open air storage spaces such as a wharf or loading bay for example may be defined as a storage space.

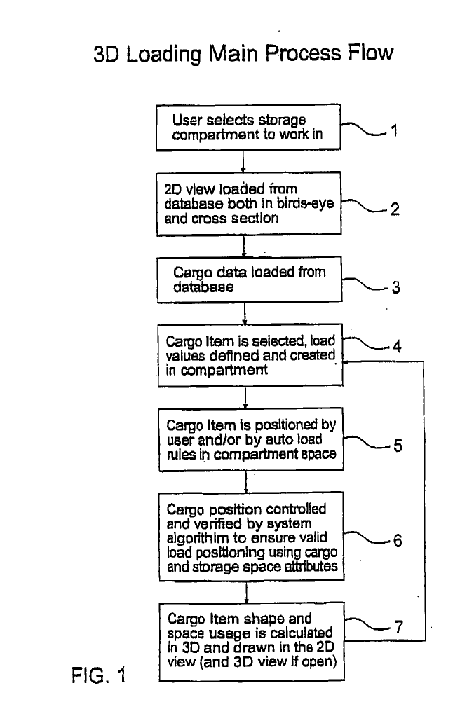

Referring to FIG. 1, an overall process flow according to the invention is shown beginning with a user selecting a storage compartment within the cargo carrying vessel to work in at step 1. This relates to a two dimensional view being loaded from a database. In the preferred embodiment there are two two dimensional views loaded, one view relating to the plan view of the storage compartment and the other view relating to the side or end view of the storage compartment. This occurs in step 2. Of course, any number of two dimensional views may be shown i.e. there could be three two dimensional views showing the “birds eye” or plan view, and the side and end views, or any combination of these views. However, providing more than one two dimensional view provides the advantage to the user that another perspective of the general shape and orientation of the cargo or cargo items within the storage space is provided. In this way the user has a better idea or understanding of the available space for other cargo. The preferred appearance of these views and the interface through which they are portrayed on a visual display unit such as a cathode ray tube or plasma screen is described with more detail below.

Once the data from the database which defines the shape or dimensions of the storage spaces has been loaded, cargo data for the cargo to be provided within that storage space, or at least cargo which is intended to be stored somewhere in the vessel, is loaded from the database in step 3.

The user then selects the cargo item that the user wishes to place within the storage space, This occurs in step 4 and when this occurs, load values arc defined and created in the storage space for the selected cargo items. The user then positions the cargo item in a desired orientation or general position in the compartment space in step 5. This step will usually be performed using a pointing device such as a “mouse” or the like. Thus a user may point to the representation of the cargo item, “click” the mouse and “drag” the item to a new location. Rather than the user manually positioning the cargo, the user may elect to allow an “auto load” function provided by the invention to be used. The auto load function has a series or rules which determine the placement of the cargo within the storage space and these rules can be set up by a user. For example, the rules may require cargo to be loaded from one side and one end of the available space, beginning at the back. This may accord with a desired physical loading or unloading procedure.

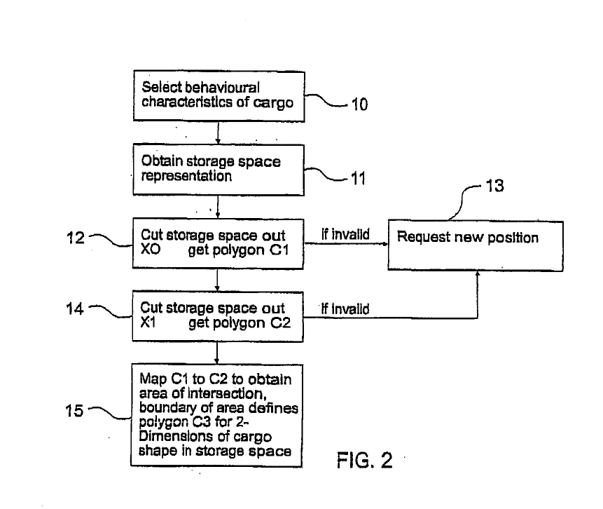

The next step relates to verification of the desired cargo position. Therefore, in step 6, the system attempts to verify the position to ensure that it is a valid load position and the system does this by performing a series of steps as outlined in the method described in FIG. 2.

In step 7, once it has been decided that the desired position of the cargo within the storage space is possible, the cargo item shape and space usage is calculated to provide a three dimensional view. The calculation is used to work out the correct shape and position for placing the cargo in the two dimensional views referred to above in step 2. Also, the three dimensional representation will be portrayed by the interface if this is elected by the user. Once step 7 is completed, the user can repeat the process again from step 4 for loading another cargo item or for changing the position of the previous cargo item.

As each item or group of items of cargo is loaded, the available storage space is correspondingly reduced. This method can thus be used to best plan how to fill the physical or real storage space with the real cargo.

Referring now to FIG. 2, in step 10 the behavioural characteristics of the cargo or the cargo items to be loaded are determined or selected. One common cargo item may be logs for example. These represent a particular problem in that they obviously cannot be bent and have a fixed length i.e. they are elongate and are best stacked in the cargo space in a single direction whereby they are all aligned. Other cargo items may comprise of containers or boxes also having predetermined dimensions, or may include particulate matter such as coal for example, which will generally conform to the overall shape of the cargo space.

Once the particular cargo item has been categorised as to its general behavioural characteristics, a representation of the storage space is obtained, for example from a database of dimensions of the storage space. This occurs in step 11.

The remainder of the method is described with reference to one particular dimension of the item to be loaded. It will be seen that the method can be applied to a number of different objects for the purposes, for example, of determining whether an object which is fixed in two or three dimensions may be placed within a given storage space, or to calculate the shape of an object which is fixed in one, two or even three dimensions within the storage space. However, for the purpose of illustration, the method will be described with reference to elongate objects such as logs. For the purposes of the method, the elongate objects are assumed to have a fixed length which is the same for each log, and to be of minimal diameter or cross section as compared to their length.

Therefore, the first step 12 is to “cut” the storage space at a point X0. The point X0 is chosen along the length of the storage space in a direction in which the cargo items, in this case a group of logs will be stacked within that space. Therefore, for example, the storage space may be thought of as extending along a general X axis parallel to which the logs will be placed within that storage space. The point X0 may be chosen as the position within the storage space along the X axis at which one end of the logs will be placed. Therefore, a cross section is taken by “cutting” through the storage space at X0 to obtain the general shape of the storage space in a plane perpendicular to the X axis at point X0. The resulting closed curve or shape which is observed in cross section at X0 is labelled C0. If the point X0 is selected to occur outside the storage space, then the method recognies that this invalid and therefore will request a new position for X0 in step 13. Depending upon the chosen position of X0 and the number of items, the shape of C0 may be dictated by the shape of the storage space.

If the position chosen for X0 is valid, then the method continues in step 14 by looking at the other end of the logs (i.e. at the other end of the chosen dimension) at a position X1 in a storage space. Therefore, the distance bets X0 and X1 will be the length of the logs. Again, the storage space is “cut” in a plane perpendicular to the X axis at point X1 to obtain a further polygon or closed curve C1. Again, if X1 is a position which is outside the storage space, then the system will recognise that this is invalid as the log simply cannot fit within the space and a new position will be requested or determined in step 13.

In step 15, the two polygons or closed curves C0 and C1 are mapped onto each other and the area of interception between those is determined. C0 and C1 will be different if a part of the storage space at X1, for example forces the logs at that point into a shape that is different to the shape at X0. The boundary of this area defines a further polygon C2 and this must be the allowable shape of the elongate elements within the storage space. This occurs in step 15. Therefore, the resultant three dimensional shape to be represented within the storage space will be the shape determined by the polygon C2 which will extend in a direction perpendicular to the polygon for a distance corresponding to the length of the logs (i.e. the distance X1-X0).

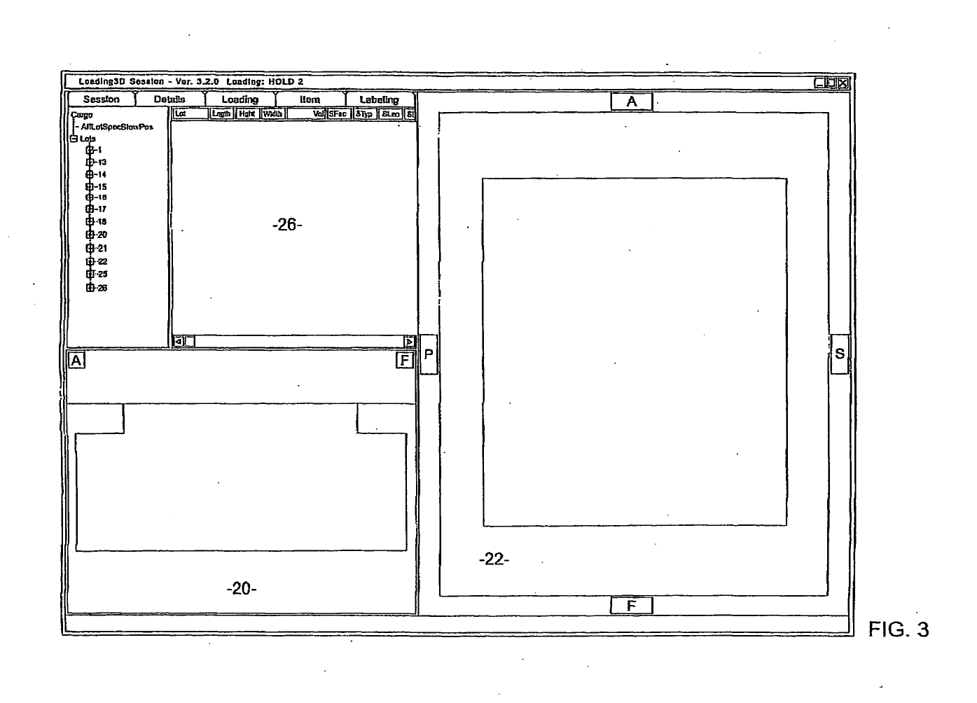

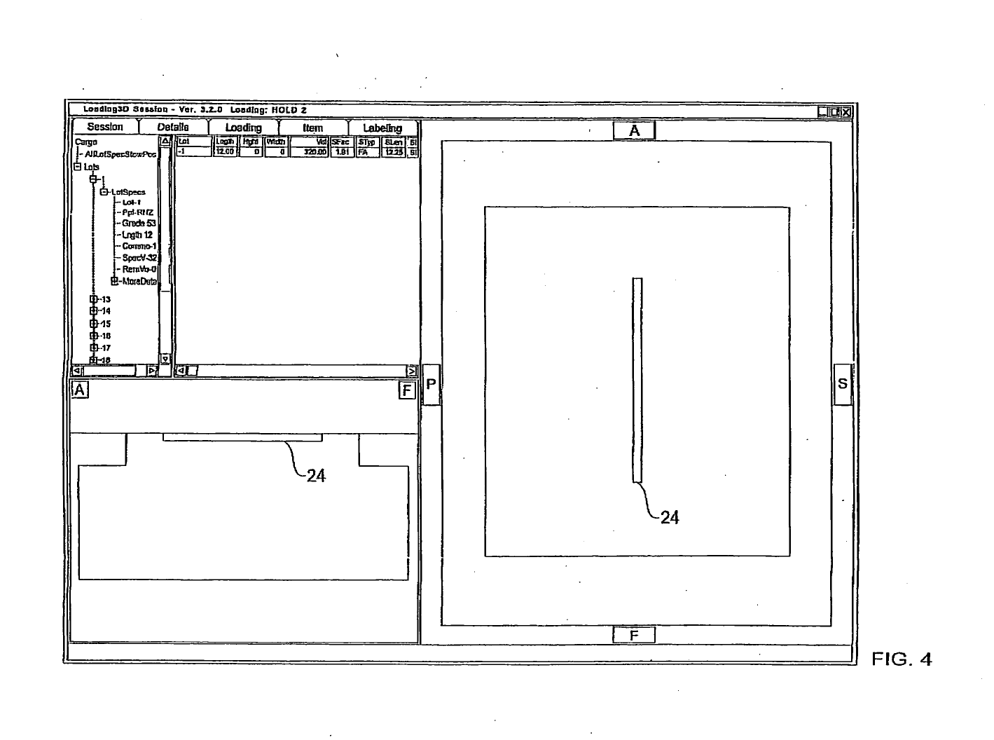

Turning now to the remaining drawing figures, these show examples of the invention in use applied to loading cargo in a ship. In FIG. 3, a first window or frame 20 shows a two dimensional view in cross section through a storage space and a second window or frame 22 shows a birds eye view of the cargo storage space. The same interface is shown in FIG. 4 showing a single cargo item 24 is shown placed at the top of the storage space. Therefore, the system allows a user to place a cargo item in free space within the hold if desired. The remaining window 26 includes data on the cargo and cargo space, as described in our co-pending application referred to earlier in this document.

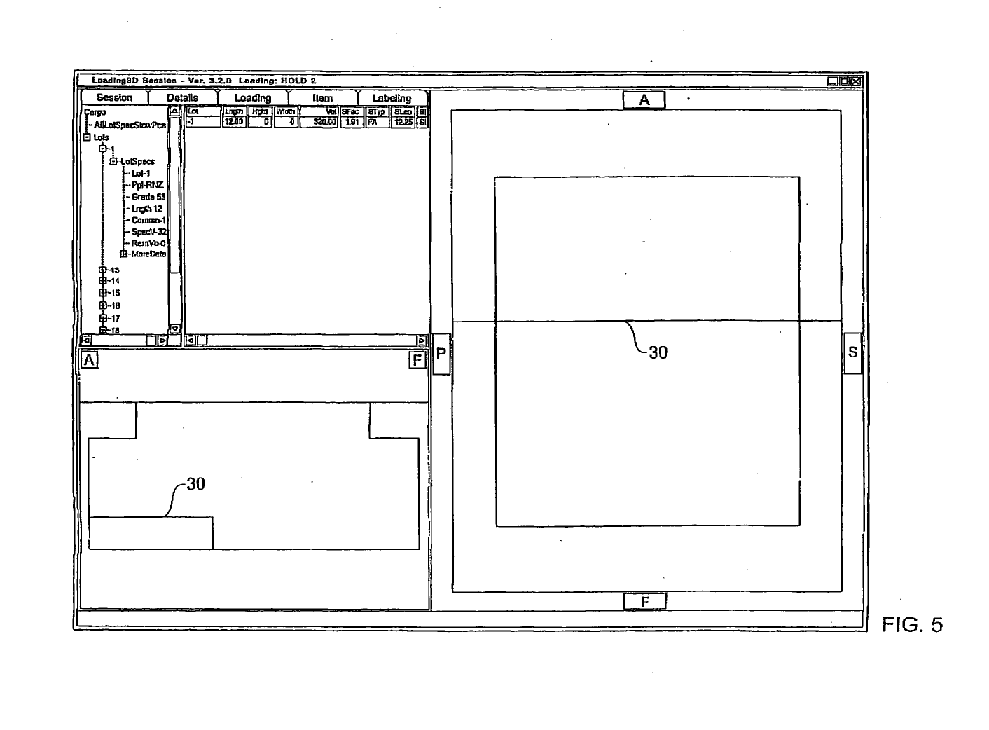

Turning to FIG. 5, a group of cargo items is shown and from the two dimensional interface in window 20, the cargo items are illustrated as a bundle of elongate items, such as logs 30, provided in the lower aft area of the storage space. Tuning to the second window 22, the two dimensional view from above illustrates that the cargo extends across the full width of the storage space (i.e. from port to starboard).

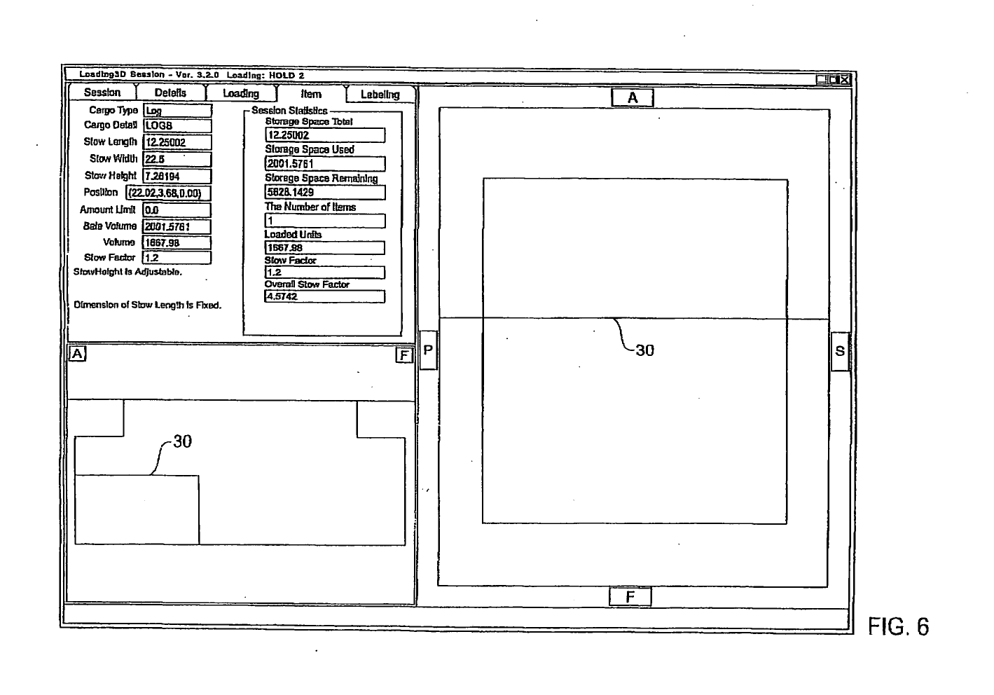

Referring to FIG. 6, the same interface as described in the preceding two figures shows a larger group of cargo items 32 in the storage space. The “item” in view that has been selected in window 26 shows that the cargo type is logs and provides additional detail regarding the length, width and height of the cargo together with volume and stow factor. Furthermore, the total storage space in used storage space is also displayed together with the overall stow factor. Although only length, width, height and stow factor are shown in this figure, the invention includes any desired units of measurement, and the units of measurement may be selected by a user. For example, the cargo may be represented by volume, weight or number of units, If the cargo is known, then a per unit measurement will enable the volume and weight to be calculated. Similar considerations apply to volume or weight measurement. The invention allows the shape and weight of each cargo item to be recorded spatially within the cargo space, for example by horizontal distance from one side and the front or back of the space, and by vertical distance from the top or bottom. With this information, known techniques arc used to calculate the centre of mass of the loaded cargo as a whole. The calculation of the cargo mass, and the centre of mass i.e. where the force exerted by the cargo acts, is then compared with known safety parameters for the cargo company vessel. Therefore, a measure of the stability of the vessel may be calculated. The invention allows stability measurements to be updated continuously as cargo is planned to be loaded or unloaded using the method and interface described. Therefore, if an item of cargo is planned to be placed in a position where stability is likely to be compromised, then a warning is provided.

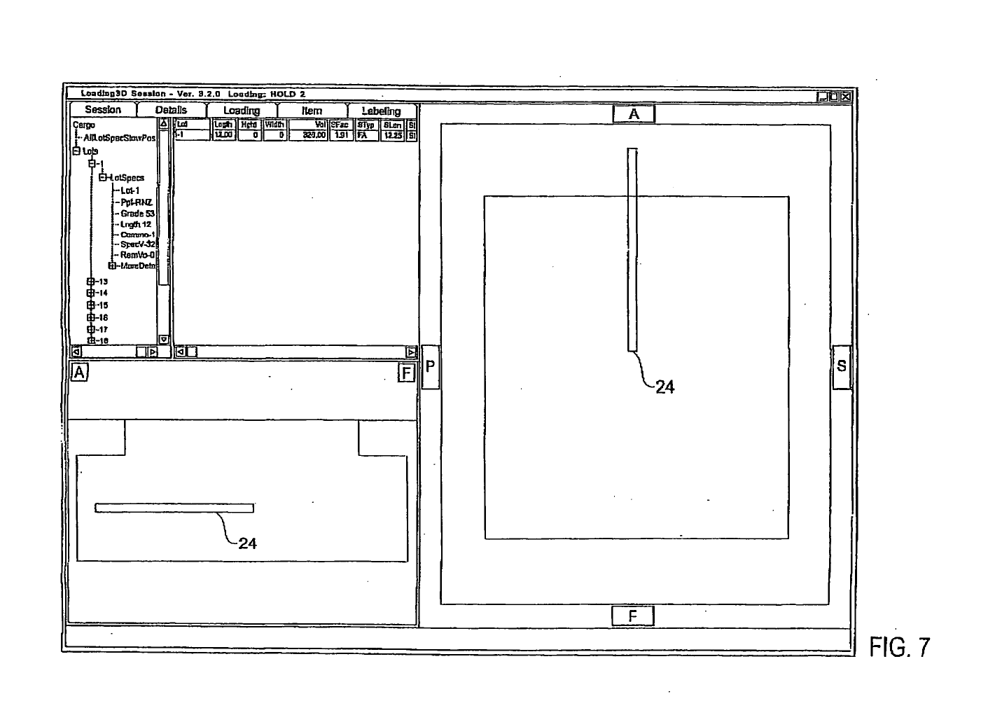

In FIG. 7, a view is shown of a cargo item being moved from a position as illustrated in the FIG. 3 into the storage space, the interface illustrating how an item of cargo is represented extending below the inner lip at the upper edge of the storage space in this example.

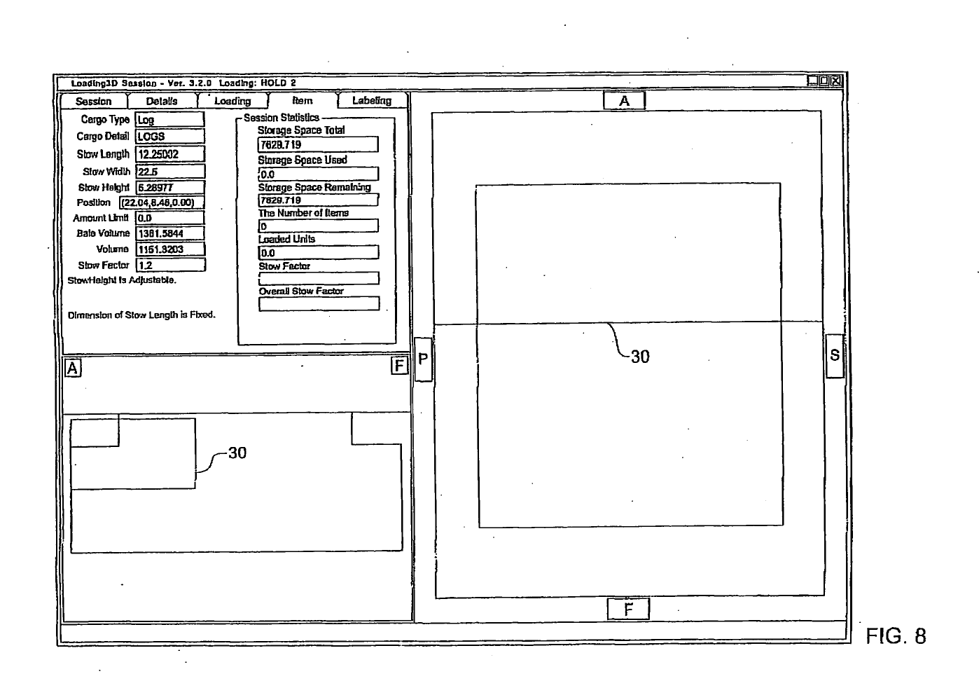

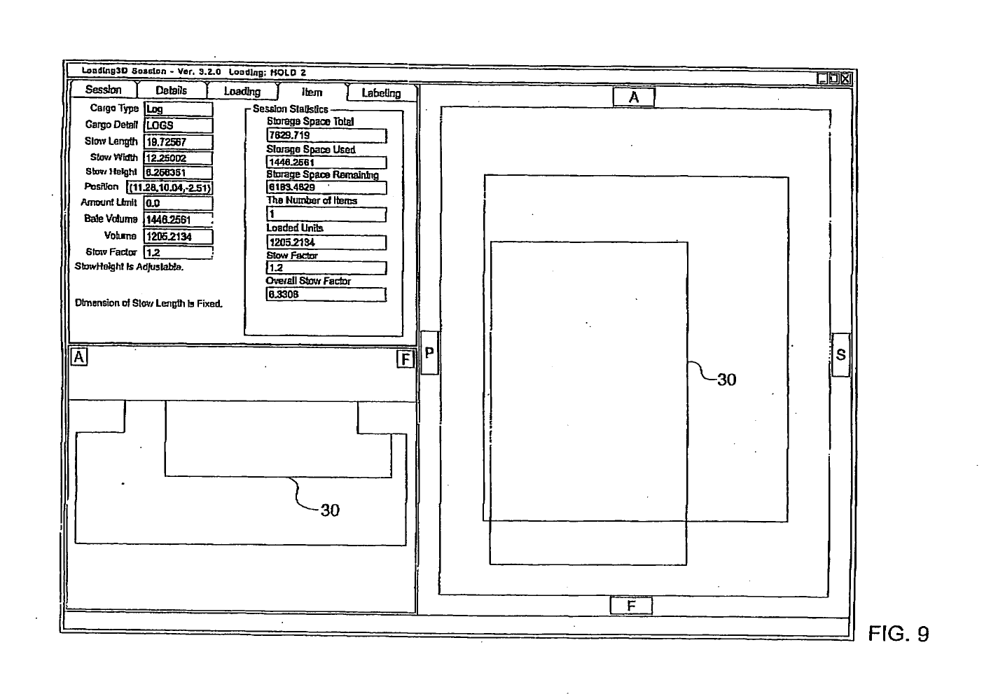

In FIG. 8 the interface illustrates the user attempting to load a group of cargo items in the upper aft part of the storage space. FIG. 8 illustrates the way in which the system responds to the request. In FIG. 8, a part of the cargo outline extends beyond the storage space area i.e. would normally extend beyond the hold if it was to be placed in the position intended by the user. In FIG. 9, the system has realized that the cargo cannot extend beyond the hold, and has redistributed the cargo so that it is shaped appropriately to fit within the storage space. As outlined previously, in order to make the appropriate decisions regarding cargo redistribution, the behavioural characteristics of the cargo needs to be taken into account. In this case the cargo is logs which have been loaded across the storage space i.e. From port to starboard and the texture shown in the two dimensional interface 20 illustrates an end view of the logs.

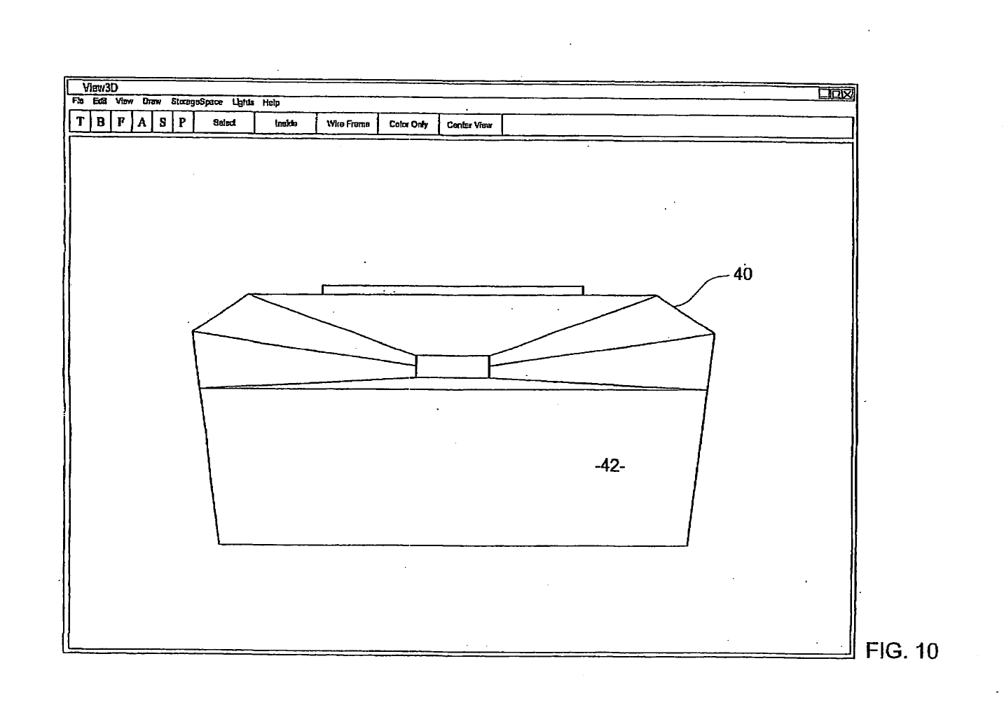

Turning now to FIG. 10, a three dimensional representation of a cargo storage space as shown, the space being referenced 40 and the cargo within that stage is represented by texture 42. This three dimensional representation may be a typical representation of a partially filled hold as shown in the two dimensional representation shown in FIG. 5.

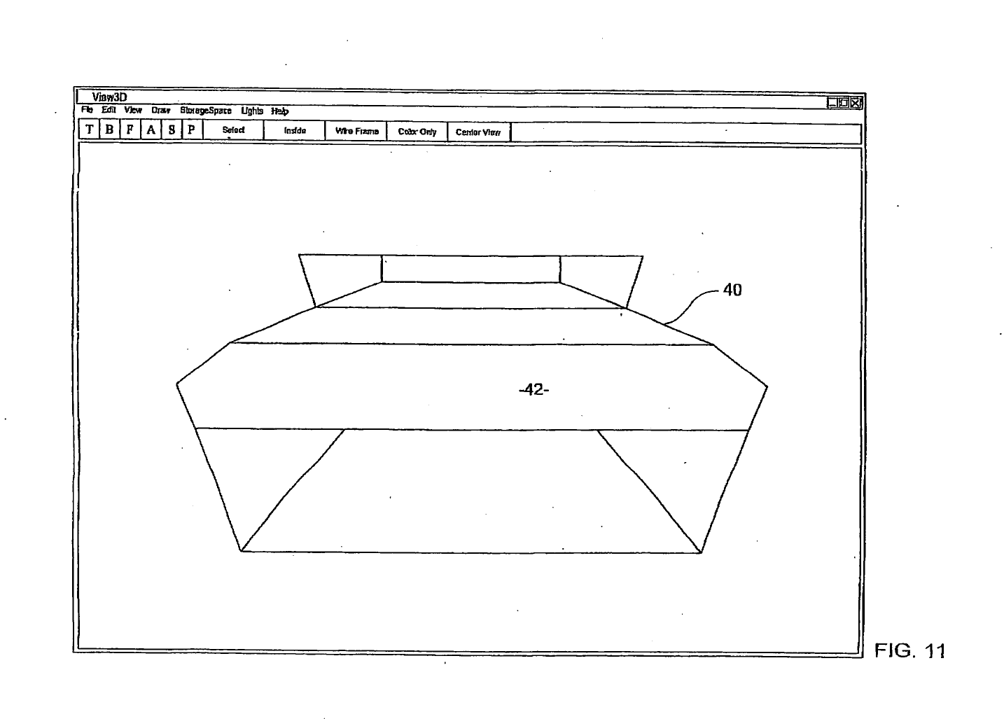

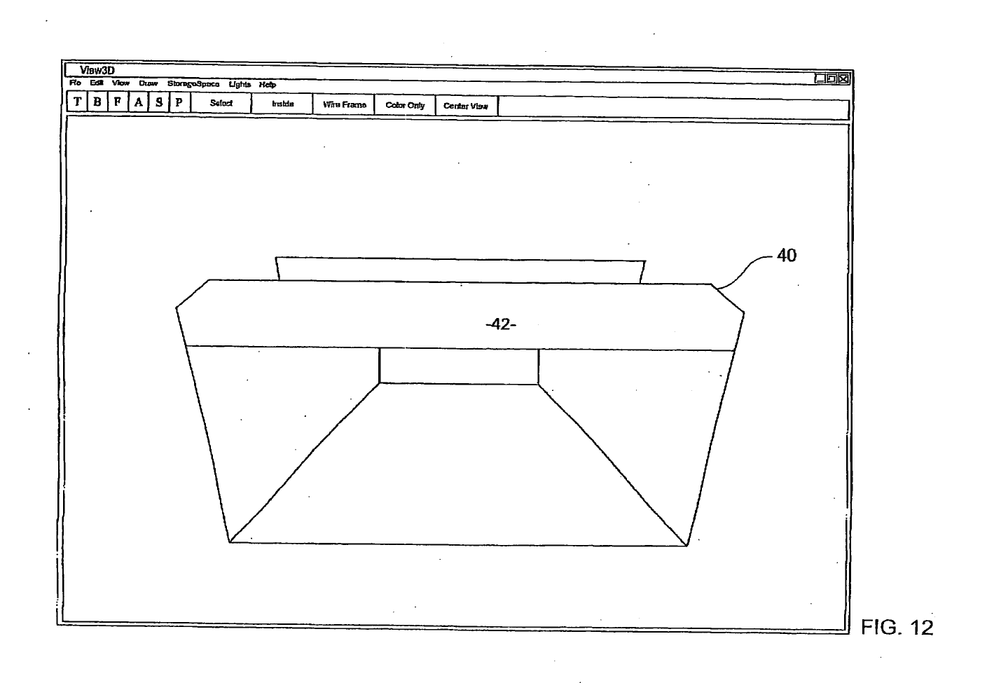

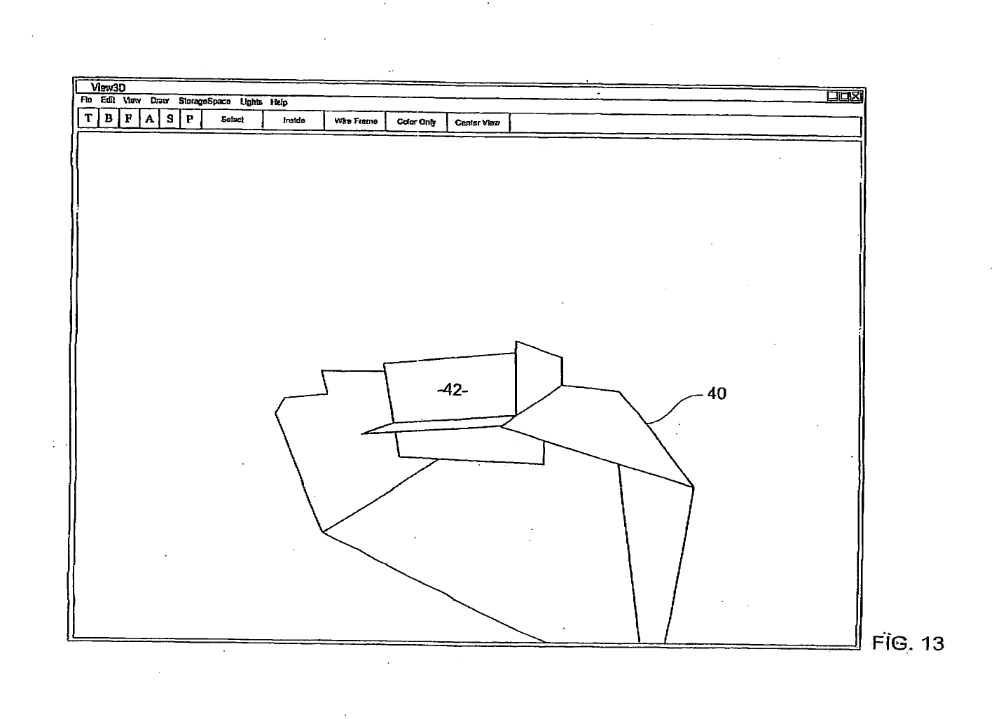

In FIG. 11, the three dimensional representation illustrates cargo placed at the top part of the storage space. Again, in FIG. 12, cargo is shown partially in and out of the storage space. In FIG. 13, a further view of the configuration of FIG. 11 is shown from a different perspective in three dimensions.

From the foregoing it will be seen that the invention provides considerable advantages in allowing a clear depiction of the cargo loading situation (or unloading situation) in a defined storage space and may be used to provide a good indication for the user of the physical amount of space available for storing cargo particularly when used in conjunction with the system of our co-pending application referred to earlier in this document. The invention also has the advantage of allowing the cargo item to be measured by object type and number of objects, by weight, or by volume. With this data, and knowing the spatial position of the item(s) within the storage space, the invention allows a stability analysis to be performed.

Such an analysis is highly beneficial to cargo carrying vessels such a aircraft, ships, trains anid road vehicles. Furthermore, rules may be implemented by the invention based on required stability, whereby cargo items of a certain size or volume may not be loaded in certain positions within the storage space for safety reasons (such as stability), or vessel performance reasons, or because of the cargo type, for example to prevent damage to adjacent cargo items.

1. A graphical user interface for indicating the location of one or more cargo items in at least one storage space, the interface including
a three dimensional representation of the storage space
a three dimensional representation of the one or more cargo items in that storage space,
whereby the representation of the one or more cargo items is able to be moved to a desired location within the representation of the storage space, and
whereby the representation of the one or more cargo items is representative of the real space required for locations of the one or more cargo items in the real storage space.

2. An interface as claimed in claim 1, wherein a plurality of cargo items are present, and the cargo items are represented as a collective shape.

3. An interface as claimed in claim 2, wherein the plurality of cargo items may be moved as a group within the representation of the storage space.

4. An interface as claimed in claim 3, wherein the shape of the group of cargo items is represented to conform to the representation of the storage space dependent upon the physical properties of the group of cargo items.

5. An interface as claimed in one of the preceding claims further including a two dimensional representation of the cargo storage space and the one or more cargo items.

6. An interface as claimed in claim 5 including two two dimensional representations, one two dimensional representation being selected from a plan, end or side view, and the other representation being selected from another of a plan, side or end view.

7. An interface as claimed in any preceding claim wherein the one or more cargo items comprises items of at least one fixed dimension.

8. A method for providing a indication of the predicted stability of a vessel when loaded with one or more cargo items, the method including the steps of determining the spatial
